(19) United States
(12) Patent Application Publication
(10) Pub. No.: US 2014/0127311 A1
(43) Pub. Date: May 8, 2014

(54) BIO-COMPATIBLE NANO-POLYMER PARTICLES COMPRISING ACTIVE INGREDIENTS FOR PULMONARY APPLICATION

(71) Applicant: JUSTUS-LIEBIG-UNIVERSITAET GIESSEN, Giessen (DE)

(72) Inventors: Moritz Beck-Broichsitter, Marburg (DE); Thomas Schmehl, Giessen (DE); Tobias Gessler, Wettenberg (DE); Thomas Kissel, Staufen (DE)

(73) Assignee: JUSTUS-LIEBIG-UNIVERSITAET GIESSEN, Giessen (DE)

(21) Appl. No.: 14/122,019

(22) PCT Filed: May 23, 2012
(86) PCT No.: PCT/EP2012/059577

§ 371 Date: Jan. 9, 2014

(30) Foreign Application Priority Data
May 25, 2011 (EP) 11167486.7

(51) Int. Cl. A61K 9/14; A61K 31/519

(52) U.S. Cl. CPC A61K 9/146; A61K 31/519
USPC 424/501; 514/772.2; 514/252.16

(57) ABSTRACT

The present invention provides biocompatible nano-polymer particles which are composed of a biocompatible polymer, a stabilizer and an active agent for the treatment of pulmonary hypertension or erectile dysfunction and which can be used to produce a pharmaceutical preparation for the treatment of pulmonary hypertension or erectile dysfunction. Biocompatible nano-polymer particles of this invention have a diameter ranging from 10 nm to 10 μm auf, a stabilizing layer thickness between 0 and 50 nm, contain between 0 and 50% of an active agent for the treatment of pulmonary hypertension or erectile dysfunction, are nebulizable and continuously release the active agent over a period of up to 48 hours. Biocompatible nano-polymer particles of this invention can be synthesized for example using the emulsion technique known to the expert with subsequent solvent evaporation or via spray drying.

The present invention provides biocompatible nano-polymer particles with active agents against pulmonary hypertension or erectile dysfunction which are suitable for pulmonary administration to treat pulmonary hypertension or erectile dysfunction in humans. The biocompatible nano-polymer particles possess the nebulization properties required for pulmonary administration and allow the targeted, controlled, sustained and long-lasting release of the active agents used.

### DESCRIPTION OF THE GENERAL FIELD OF INVENTION

The present invention concerns the fields of internal medicine, pharmacology, nanotechnology and medical technology.

### STATE OF THE ART

The specific drug therapy of pulmonary hypertension and erectile dysfunction primarily comprises the intravenous or oral administration of potent vasodilators. Pulmonary hypertension is a serious, life-threatening disorder which substantially limits physical capacities. The increase of pulmonary artery pressure and vascular resistance with subsequent dysfunction of the right heart results in a severely reduced life expectancy with an average survival time of only 2.8 years after diagnosis without treatment.

Counted among vasodilators is for example the class of phosphodiesterase inhibitors. Phosphodiesterases are responsible for the degradation of the intracellular transmitters (second messenger) cyclic adenosine monophosphate (cAMP) and cyclic guanosine monophosphate (cGMP). Phosphodiesterase-5 is able to selectively break down cGMP. cGMP is the second messenger which is activated by the endothelial relaxation factor nitrogen monoxide (NO) and involved in the relaxation of blood vessels. Since phosphodiesterase-5 inhibitors inhibit the inactivation of cGMP, these inhibitors lead to an enhancement of the vasodilating effect of nitrogen monoxide. Phosphodiesterase-5 inhibitors were originally developed for the treatment of angina pectoris, are however today primarily used for the therapy of erectile dysfunction and pulmonary hypertension. The effect of phosphodiesterase-5 inhibitors becomes particularly evident in tissues with high expression of phosphodiesterase-5. These are in addition to the smooth musculature of systemic and pulmonary blood vessels, where phosphodiesterase-5 inhibitors cause a relaxation, also immunocompetent cells and thrombocytes.

One active agent of the group of phosphodiesterase-5 inhibitors for the treatment of pulmonary hypertension and erectile dysfunction is sildenafil which is administered to the patient orally tree times daily as sildenafil-citrate (Revatio®). The oral administration of sildenafil however results in a systemic availability of the drug which is associated with significant side effects. Other phosphodiesterase inhibitors are phosphodiesterase-3 inhibitors and phosphodiesterase-4 inhibitors.

Phosphodiesterase-3 inhibitors (PDE-3 inhibitors) are a subgroup of medicaments of the group of phosphodiesterase inhibitors which are approved for the therapy of acute cardiac insufficiency with lacking response to catecholamines due to a down-regulation of receptors at the myocard. Drugs approved so far are amrinon, cilostazol, milrinon and enoximon. The active compound pimobendan is approved for an application in dogs. An inhibition of phosphodiesterase-3 results in an increase of the second messenger cAMP. PDE-3 inhibitors furthermore exhibit a vasodilating effect.

Phosphodiesterase-4 inhibitors (PDE-4 inhibitors) are substances which inhibit the enzyme phosphodiesterase-4. Phosphodiesterase-4 breaks down the second messenger cAMP and cGMP. PDE-4 inhibitors thus increase the concentration of intracellular cGMP (cyclic guanosine monophosphate). The enzyme is among others present in the lung. The archetype of phosphodiesterase-4 inhibitors is rolipram. PDE-4 inhibitors have an anti-inflammatory effect and were investigated among others for an application in COPD, asthma bronchiale, depression and multiple sclerosis. Until today, only one active agent has been approved as drug: roflumilast (Daxas®).

Further active agents for the treatment of pulmonary hypertension are activators and stimulators of the soluble guanylate cyclase. To this group of activators belong for example cinaciguat and ataciguat; to the group of stimulators belong for example riociguat, BAY41-2272, BAY41-8543 and CFM-1571.

Beyond this, also endothelin receptor antagonists are used for the treatment of pulmonary hypertension; these are e.g. bosentan, zibotentan, tezosentan, macitentan, sitaxentan, avosentan, clazosentan, ambrisentan, darusentan, atrasentan, enrasentan.

Other active agents for the treatment of pulmonary hypertension are prostanoids; among these count for example prostacyclin, treprostinil and iloprost.

Prior art knows of inhalations as a more selective route of administration by which undesired systemic side effects can be avoided. The direct administration of the drug to the lung facilitates the targeted treatment of respiratory diseases as already demonstrated for the prostacyclin-analogue iloprost (Ventavis®) in the treatment of pulmonary hypertension. The relatively short duration of pharmacological effects after pulmonary drug deposition however is a major disadvantage of inhalation therapy, requiring a frequent drug administration via inhalation and leading to therapeutic gaps particularly during the night.

Colloidal materials such as e.g. biocompatible nano-polymer particles are known as suitable pulmonary drug delivery systems. With a direct delivery of therapeutic agents which are encapsulated in biocompatible nano-polymer particles into the lung, a prolonged and controlled drug release can be achieved at the desired target site, thus resulting in a prolongation of pharmacological effects.

The choice of the production method substantially depends on the physicochemical parameters of the polymer used, as well as from the active agent to be encapsulated in biocompatible nano-polymer particles. The choice of the polymer is determined by criteria such as biocompatibility and biodegradability. In addition, biocompatible nano-polymer particles have to meet further standards such as for example a sufficient association of the therapeutic agent with the carrier material as well as a sufficiently high stability against forces generated during nebulization. These stringent requirements are met by nanoparticulate drug delivery systems composed of biocompatible polymers.

The solvent evaporation technique (evaporation method) is known to be a suitable preparation method for biocompatible nano-polymer particles. This method comprises the emulsification of an organic polymer solution into an aqueous phase containing a stabilizing excipient. Even though prior art knows that the employed stabilizers modulate the physicochemical and biological properties of biocompatible nano-polymer particle formulations used, the exact relevance of these formulation parameters for the aerodynamic properties of nebulized formulations and for biocompatible nano-polymer particle stability is still unknown.

Summarizing, the state of the art discloses suitable active agents for the treatment of pulmonary hypertension or erectile dysfunction, whose pharmacological effect however is only very short in the case of a pulmonary administration and/or associated with significant side effects if administered systemically (orally, subcutaneously, intravenously etc.). The state of the art is furthermore disadvantageous with regard to the aerodynamic properties and stability of nebulized biocompatible nano-polymer particle formulations.

### Aim

Aim of the present invention is to provide an aerosolizable and inhalable pharmaceutical preparation for the treatment of pulmonary hypertension or erectile dysfunction which contains an active agent for pulmonary hypertension or erectile dysfunction, allows a long-lasting and controlled release of the active agent, and is suitable for an application in humans.

### Solution of the Aim

The aim to provide a pharmaceutical preparation for the treatment of pulmonary hypertension or erectile dysfunction is solved according to the present invention by biocompatible nano-polymer particles composed of a biocompatible polymer and a stabilizer as well as an active agent chosen from the group of phosphodiesterase inhibitors (PDE inhibitors) or guanylate cyclase activators or guanylate cyclase stimulators or endothelin receptor antagonists or the prostanoids.

Biocompatible nano-polymer particles of the present invention can be prepared using the emulsion method with subsequent solvent evaporation. The thin protective stabilizer films which consist for example of polyvinyl alcohol (PVA) and are formed on the biocompatible nano-polymer particles of this invention improve the stability of particles during nebulization. The suspension of biocompatible nano-polymer particles of this invention can be converted into an aerosol which is suitable for a deposition in the lung. Physicochemical characteristics of biocompatible nano-polymer particles of this invention (e.g. size, surface charge, drug loading etc.) are not influenced by the nebulization process. The prolonged drug release achieved with this new pulmonary drug transport system for active agents for pulmonary hypertension results in a reduced frequency of medication as compared to conventionally applied pharmaceutical compositions, thus improving life quality and compliance of patients. Summing up, biocompatible nano-polymer particles of this invention are a promising therapeutic agent for the treatment of pulmonary hypertension or erectile dysfunction.

Biocompatible nano-polymer particles of the present invention are composed of a biocompatible polymer as well as a stabilizer and an active agent for the treatment of pulmonary hypertension or erectile dysfunction which is chosen from the group of phosphodiesterase inhibitors (PDE inhibitors) or guanylate cyclase activators or guanylate cyclase stimulators or endothelin receptor antagonists or the prostanoids. The biocompatible polymer is for example a polyester, polyanhydride, polyorthoester, polyphosphoester, polycarbonate, polyketal, polyurea, polyurethane, block copolymer (PEG-PLGA), star polymer or comb polymer.

The polyester is preferably a linear poly(lactide-co-glycolide) copolymer (PLGA copolymer). The comb polymer is preferably a charge-modified branched poly(vinyl sulfonate-co-vinyl alcohol)-graft-poly(D,L-lactide-co-glycolide) copolymer (P(VS-VA)-g-PLGA) or sulfobutyl-polyvinyl alcohol-graft-poly(lactide-co-glycolide) copolymer (SB-PVA-g-PLGA).

For the preparation of biocompatible nano-polymer particles, suitable PLGA polymers exist which are used for a controlled release of the active agent. These comprise for example, but not exhaustively, copolymers of the Resomer®-family. In a preferred embodiment of the invention, biocompatible nano-polymer particles contain one of the following Resomer® substances Resomer® Condensate RG 50:50 Mₙ 2300, Resomer® R202S, Resomer® R202H, Resomer® R203S, Resomer® R203H, Resomer® R207S, Resomer® RG502H, Resomer® RG503H, Resomer® RG504H, Resomer® RG502, Resomer® RG503, Resomer® RG504, Resomer® RG653H, Resomer® RG752H, Resomer® RG752S, Resomer® RG753S, Resomer® RG755S, Resomer® RG756S or Resomer® RG858S. In a particularly preferred embodiment, biocompatible nano-polymer particles of the present invention contain the PLGA copolymer Resomer® RG502H.

Suitable P(VS-VA)-g-PLGA copolymers for the preparation of biocompatible nano-polymer particles are for example P(VS-VA)-g-PLGA 2-10, P(VS-VA)-g-PLGA 4-10, P(VS-VA)-g-PLGA 6-5, P(VS-VA)-g-PLGA 6-10, P(VS-VA)-g-PLGA 6-15 or P(VS-VA)-g-PLGA 8-10.

The state of the art furthermore also describes appropriate stabilizers which can be used for the preparation of biocompatible nano-polymer particles suitable for a controlled drug release. According to the present invention, the stabilizer is chosen from the group of non-ionic surfactants, anionic surfactants, amphoteric surfactants or the polymers. Non-ionic surfactants are for example, but not exhaustively, tween, span or pluronic. An anionic surfactant is for example, but not exhaustively, sodium dodecyl sulfate (SDS), an amphoteric surfactant is for example, but not exhaustively, lecithin. Suitable polymers are for example the hydrophilic polymers polyethylene glycol (PEG), polyethyleneimine (PEI), polyvinyl alcohol (PVA), polyvinyl acetate, polyvinyl butyrate, polyvinylpyrrolidone (PVP) or polyacrylate as well as natural polymers such as proteins (e.g. albumin), celluloses and esters and ethers thereof, amylose, amylopectin, chitin, chitosan, collagen, gelatin, glycogen, polyamino acids (e.g. polylysine), starch, modified starches (e.g. HES), dextrans or heparins.

In a preferred embodiment, biocompatible nano-polymer particles contain polyvinyl alcohol, hereinafter abbreviated as PVA, as stabilizer. PVA is a crystalline, water-soluble plastic material.

Biocompatible nano-polymer particles of the present invention furthermore contain an active agent for pulmonary hypertension or erectile dysfunction, chosen from the group of phosphodiesterase inhibitors (PDE inhibitors) or guanylate cyclase activators or guanylate cyclase stimulators or endothelin receptor antagonists or the prostanoids. PDE inhibitors which are suitable for the treatment of pulmonary hypertension and erectile dysfunction are among others the phosphodiesterase-5 inhibitors (PDE-5 inhibitors). PDE-5 inhibitors are agents which inhibit the cGMP-degrading enzyme phosphodiesterase 5 (PDE-5) and therefore increase the concentration of intracellular cGMP (cyclic guanosine monophosphate). Among others, they cause a dilation of blood vessels (vasodilation). Based on the selectivity for phosphodiesterase isoform 5, non-selective phosphodiesterase inhibitors such as the methylxanthines caffeine, theophylline, theobromine, which unspecifically inhibit different phosphodiesterases, can be distinguished from selective inhibitors of phosphodiesterase-5 such as for example the active agents sildenafil, tadalafil and vardenafil. In a preferred embodiment, biocompatible nano-polymer particles of this invention contain sildenafil as active agent. To those skilled in the art, sildenafil is also known under the chemical formula 5-[2-ethoxy-5-(4-methyl-1-piperazinyl sulphonyl)phenyl]-1-methyl-3-n-propyl-1,6-dihydro-7H-pyrazolo[4,3-d]pyrimidine-7-one. In a particularly preferred embodiment, free base sildenafil is concerned.

In addition to PDE-5 inhibitors, also phosphodiesterase-3 inhibitors (PDE-3 inhibitors) and phosphodiesterase-4 inhibitors (PDE-4 inhibitors) belong to the PDE inhibitors. PDE-3 inhibitors are used for the therapy of acute cardiac insufficiency with lacking response to catecholamines. An inhibition of phosphodiesterase-3 results in an increase of the second messenger cAMP. PDE-3 inhibitors furthermore exhibit a vasodilating effect. PDE-4 inhibitors are agents inhibiting the enzyme phosphodiesterase-4 which breaks down the second messenger cAMP and cGMP. PDE-4 inhibitors therefore increase the concentration of intracellular cGMP (cyclic guanosine monophosphate). The enzyme is among others present in the lung. PDE-4 inhibitors have an anti-inflammatory effect.

Other suitable active agents for the treatment of pulmonary hypertension are guanylate cyclase activators or guanylate cyclase stimulators and endothelin receptor antagonists.

Guanylate cyclase activators are for example cinaciguat and ataciguat; guanylate cyclase stimulators are riociguat, BAY41-2272, BAY41-8543 and CFM-1571.

Endothelin receptor antagonists are for example bosentan, zibotentan, tezosentan, macitentan, sitaxentan, avosentan, clazosentan, ambrisentan, darusentan, atrasentan, enrasentan.

Further suitable agents for the treatment of pulmonary hypertension are prostanoids, among which are counted for example prostacyclin, treprostinil and iloprost.

Characterization of biocompatible nano-polymer particles of this invention

Biocompatible nano-polymer particles of this invention have a mean geometric diameter ranging from 10 nm to 10 μm, so that they are well nebulizable, and a stabilizing layer thickness between 0 and 50 nm. The stabilizing layer thickness however does not exceed the mean geometric radius of the biocompatible nano-polymer particles. In a preferred embodiment, biocompatible nano-polymer particles have a mean geometric diameter between 500 nm and 5 μm to allow a longer-lasting drug release, or a mean geometric diameter between 50 nm and 250 nm in order to prevent an uptake of particles by macrophages.

Furthermore, biocompatible nano-polymer particles of this invention preferably have a negative surface charge and a negative zeta potential. Alternatively, biocompatible nano-polymer particles may also have a positive surface charge and a positive zeta potential.

According to the present invention, biocompatible nano-polymer particles contain between 0 and 50% (w/w), and in a preferred embodiment between 1 and 20% (w/w) of an active agent for the treatment of pulmonary hypertension or erectile dysfunction.

Biocompatible nano-polymer particles of this invention are preferably nebulizable with piezoelectric, jet-, ultrasound aerosol generators, soft-mist inhalers, metered dose inhalers or dry powder inhalers, i.e. the delivery to the lung is performed via inhalation of an aerosol (suspension, powder) using an aerosol generator. Another route of administration to the lung is via instillation, for example using a catheter, a bronchoscope or a respiratory therapy device (e.g. tube or tracheal cannula).

Preparation of biocompatible nano-polymer particles of this invention

Biocompatible nano-polymer particles of the present invention are for example synthesized using the emulsion method and subsequent solvent evaporation (evaporation method). Biocompatible nano-polymer particles of this invention are composed of a biocompatible polymer and as well as a stabilizer and an active agent for the treatment of pulmonary hypertension or erectile dysfunction. The biocompatible polymer of said nano-polymer particles is for example a polyester (PLGA, PLA), polyanhydride, polyorthoester, polyphosphoester, polycarbonate, polyketal, polyurea, polyurethane, block copolymer (PEG-PLGA), star polymer or comb polymer. According to the present invention, the stabilizer is chosen from the group of non-ionic surfactants, anionic surfactants, amphoteric surfactants or the polymers. The active agent of biocompatible nano-polymer particles of this invention is chosen from the group of phosphodiesterase inhibitors (PDE inhibitors) or guanylate cyclase activators or guanylate cyclase stimulators or endothelin receptor antagonists or the prostanoids.

Alternatively, biocompatible nano-polymer particles are also prepared using nano-precipitation, salting-out, polymerization or spray drying. These mentioned preparation procedures are known to the expert in this field.

If particles are prepared using the evaporation method, the polymer is initially dissolved in a solvent with addition of an active agent for the treatment of pulmonary hypertension or erectile dysfunction. The concentration of the active agent employed is thereby between 7% and 20% related to the polymer to obtain a theoretical particle drug loading of 5%. Subsequently, the organic phase is transferred into a constant volume of aqueous phase containing a stabilizer. After mixing both phases and sonication with ultrasound, the organic solvent is subsequently removed by evaporation and the particles in suspension are obtained. Suitable solvents in which the polymer used according to the present invention is soluble to at least 0.1% (w/w) are for example, but not exhaustively, dichloromethane, chloroform, ethyl acetate, benzyl alcohol, methyl ethyl ketone, propylene carbonate. In a preferred embodiment, polyvinyl alcohol (PVA) is used as stabilizer.

In a preferred embodiment, biocompatible polymer between 1 and 100 g/l and stabilizer between 0.1 and 25 g/l is used for the preparation of biocompatible nano-polymer particles of this invention. In a particularly preferred embodiment, the biocompatible polymer concentration is 50 g/l and the stabilizer concentration is 10 g/l for the preparation.

In the case that the active agent used is the PDE-5 inhibitor sildenafil, the preparation of biocompatible nano-polymer particles is carried out in the presence of sildenafil in an aqueous phase at a pH value between 2 and 10. Sildenafil is an amphoteric compound with a pH-dependent solubility profile and limited solubility at neutral pH values.

An alternative preparation method for biocompatible nano-polymer particles of this invention is spray drying. For this purpose, between 0.1% and 10% of the polymer with or without addition of between 1% and 20% (related to the polymer used) of an active agent for pulmonary hypertension or erectile dysfunction like for example the PDE-5 inhibitor sildenafil is dissolved in a water-immiscible solvent such as e.g. methylene chloride. After filtration, this solution is then spray-dried with a spray dryer, for example a nano spray dryer, as specified by the manufacturer. The spray drying procedure using a spray dryer may alternatively also follow after production steps of the emulsion method with subsequent solvent evaporation, of nano-precipitation, of salting-out, or of polymerization.

Utilization of biocompatible nano-polymer particles of this invention

Biocompatible nano-polymer particles of this invention can be used for the manufacture of a pharmaceutical composition for the treatment of pulmonary hypertension or erectile dysfunction. The term biocompatibility thereby means compatibility for tissue and cells at the target site, e.g. the lung.

The effect of biocompatible nano-polymer particles of this invention is based on the active agent for the treatment of pulmonary hypertension or erectile dysfunction contained therein, which is released from the biocompatible nano-polymer particles in a controlled, continuous and long-lasting manner over a period of up to 48 hours in the lung or the bronchi or the lung interfaces.

All features and advantages illustrated in the claims, the description and the figures, including design details, spatial arrangement and process steps, may be essential to the invention, either independently by themselves as well as combined with one another in any form.

### EMBODIMENTS

The following embodiments 1 and 2 describe respectively the preparation and characterization of biocompatible nano-polymer particles of this invention. In these embodiments, the PDE-5 inhibitor sildenafil is used as active agent and is accordingly to be considered as example for a PDE-5 inhibitor. Biocompatible nano-polymer particles of the present invention are hereinafter in short referred to as particles. Poly(D,L-lactide-co-glycolide) copolymer (PLGA) or poly(vinyl sulfonate-co-vinyl alcohol)-graft-poly(D,L-lactide-co-glycolide) copolymer (P(VS-VA)-g-PLGA) is hereinafter also in short referred to as polymer.

### 1. Embodiment 1

### 1.1. Preparation of Biocompatible Nano-Polymer Particles of the Present Invention According to Claim 14 Using Emulsion and Subsequent Evaporation

Biocompatible nano-polymer particles of this invention are for example prepared at room temperature using the emulsion method with subsequent solvent evaporation which is known in the art. For this, between 1 and 100 g/l poly(D,L-lactide-co-glycolide) copolymer (PLGA), which is commercially available and can for example be obtained as Resomer® RG502H, RG502, RG503H or RG504H from Boehringer Ingelheim (Ingelheim, Germany), or poly(vinyl sulfonate-co-vinyl alcohol)-graft-poly(D,L-lactide-co-glycolide) copolymer (P(VS-VA)-g-PLGA) are initially dissolved with or without addition of between 1% and 20% of an active agent for the treatment of pulmonary hypertension or erectile dysfunction like for example the PDE-5 inhibitor sildenafil, which is commercially available as free base and provided for example by AK Scientific (Mountain View, Calif., USA), in a water-immiscible solvent like for example methylene chloride. To achieve a theoretical drug loading of 5% of the biocompatible nano-polymer particles of this invention, between 7% and 20% of the active agent is used, related to the polymer utilized. Then, 2 ml of the organic phase (dispersed phase) are transferred into 10 ml of an aqueous phase (constant phase) adjusted to pH 8 for example with 1 mM 4-(2-hydroxyethyl)piperazine-1-ethanesulfonic acid which contains between 0.1 and 15 g/l of a surface stabilizer, for example polyvinyl alcohol (PVA). PVA is commercially available for example as Mowiol 4-88® provided by Sigma-Aldrich (Steinheim, Germany). After mixing both phases, the emulsion is sonicated. Subsequently, the organic phase is slowly removed by solvent evaporation in a rotary evaporator. The particles are utilized immediately after preparation. Alternatively, the preparation procedure is followed by spray drying of biocompatible nano-polymer particles of this invention. For this purpose, biocompatible nano-polymer particles of this invention (0.2% to 2%) are spray-dried after filtration using a spray dryer like for example the Nano Spray Dryer B-90 (Büchi, Flawil, Switzerland) according to the manufacturer's instructions.

1.2 Preparation of Biocompatible Nano-Polymer Particles of this Invention According to Claim 15 Using Spray Drying

Biocompatible nano-polymer particles of this invention are for example prepared using spray drying. For this, between 0.1% and 10% poly(D,L-lactide-co-glycolide) copolymer (PLGA) which is commercially available and can for example be obtained as Resomer® RG502H from Boehringer Ingelheim (Ingelheim, Germany) is dissolved with or without addition of between 1% and 20% (related to the polymer used) of an active agent for the treatment of pulmonary hypertension or erectile dysfunction like for example the PDE-5 inhibitor sildenafil, which is commercially available as free base and provided for example by AK Scientific (Mountain View, Calif., USA), in a water-immiscible solvent like for example methylene chloride. After filtration, this solution is then spay-dried using a spray dryer like for example the Nano Spray Dryer B-90 (Büchi, Flawil, Switzerland) according to the manufacturer's instructions.

### 2. Embodiment 2

Characterization of Biocompatible Nano-Polymer Particles of this Invention

Biocompatible nano-polymer particles prepared according to Embodiment 1.1 or 1.2 are characterized using methods and results as described below under Embodiment 2, items 2.1 to 2.4. For this purpose, biocompatible nano-polymer particles are either utilized directly after preparation or after nebulization with a nebulizer, for example Aeroneb® Professional provided by Aerogen (Dangan, Galway, Ireland), as specified by the manufacturer.

2.1 Diameter, Size Distribution and Surface Charge of Biocompatible Nano-Polymer Particles of this Invention

Freshly prepared biocompatible nano-polymer particles which are synthesized using the emulsion method with subsequent solvent evaporation as described in Embodiment 1.1 are investigated in various combinations of polymer concentration (ranging from 5 to 100 g/l) and PVA concentration (ranging from 1 to 50 g/l) with respect to their properties diameter, size distribution and surface charge. Hydrodynamic diameter and size distribution (polydispersity, PDI) of biocompatible nano-polymer particles are measured with dynamic light scattering (DLS). The zeta potential as a measure for the surface charge is determined by laser Doppler anemometry (LDA), for example with a zetasizer NanoZS/ZEN3600 (Malvern Instruments, Herrenberg, Germany). All measurements are carried out at a temperature of 25° C. with aliquots appropriately diluted with filtrated and double-distilled water for DLS or with 1.56 mM NaCl for LDA. All measurements are performed at least in triplicate with at least 10 runs directly after preparation of biocompatible nano-polymer particles. In the following, n indicates the number of determinations.

A narrow particle size distribution, i.e. polydispersity indices (PDI) with a value lower than 0.1, is obtained with at a PVA concentration of more than 5 g/l at a constant PLGA concentration of 50 g/l or at a PLGA concentration between 10 and 50 g/l at a constant PVA concentration of 10 g/l. The size distribution of freshly prepared biocompatible nano-polymer particles determined via DLS is depicted in FIG. 1. The biocompatible nano-polymer particle size ranges from 100 to 400 nm (black line in FIG. 1). Biocompatible nano-polymer particles which are prepared using a PLGA concentration of 50g/l and a PVA concentration of 10 g/l have a mean particle size of 195.1±9.6 nm (mean value±standard deviation, n=4), a narrow size distribution, i.e. a small polydispersity index (PDI) of 0.078±0.002 (mean value±standard deviation, n=4) as well as a negative surface charge, i.e. a negative zeta potential of −5.7±0.8 mV (mean value±standard deviation, n=4).

To investigate the diameter, the size distribution and the surface charge as a measure for biocompatible nano-polymer particle stability after nebulization, biocompatible nano-polymer particles of this invention are prepared with a theoretical content of 5% (w/w) of the active agent sildenafil (free base) according to Embodiment 1.1 and characterized before and after nebulization using the nebulizer Aeroneb® Professional. For this, nebulized suspensions of biocompatible nano-polymer particles are collected and assessed qualitatively as described by Dailey et al. (Dailey L A, Kleemann E, Wittmar M et al.: Surfactant-free, biodegradable nanoparticles for aerosol therapy based on the branched polyesters, DEAPA-PVAL-g-PLGA. Pharm. Res. 20(12), 2011-2020 (2003); Dailey L A, Schmehl T, Gessler T et al.: Nebulization of biodegradable nanoparticles: impact of nebulizer technology and nanoparticle characteristics on aerosol features. J. Controlled Release. 86(1), 131-144 (2003)). Suspensions of biocompatible nano-polymer particles are nebulized at an air flow rate of 5 l/min and collected by placing a glass microscope slide directly in front of the nebulizer T-shaped mouthpiece, which allows a deposition of aerosol droplets on the glass microscope slide. The resulting condensation fluid is collected for further analysis. The stability of nebulized biocompatible nano-polymer particles is assessed as described above using DLS and LDA.

Biocompatible nano-polymer particles of this invention have an average size of 197.1±1.7 nm, a narrow size distribution with a PDI of 0.074±0.005 as well as a negative surface charge with a zeta potential of −5.1±0.3 mM. The parameters particle size, PDI and sildenafil content (see 2.3) are depicted in FIG. 2 as quotient of value before and value after nebulization. The figure shows that nebulization has no significant influence on the above mentioned parameters.

### 2.2 Stabilizing Layer Thickness of Biocompatible Nano-Polymer Particles of the Present Invention

The thickness of adsorbed PVA layers serving as surface active stabilizers of biocompatible nano-polymer particles of this invention is determined using DLS- and zeta potential measurements as described under item 2.1 as a function of electrolyte concentration. Suitable assay methods are known to the expert in this field. With respect to the DLS measurements, the adsorbed PVA layer thickness (δ) is derived from comparing the particle sizes of bare (d₀) and coated (d_{ads}) biocompatible nano-polymer particles according to the following equation (1)

$$δ = \frac{{d}_{ads} - {d}_{0}}{2} \qquad ( 1 )$$

Layer thickness from zeta potential measurements is calculated using the Gouy-Chapman approximation known to the expert, which expresses the decrease of the electrostatic potential as a function of the distance from the surface in the following equation (2)

Ψₓ=Ψ₀·e^{κx} (2)

where Ψₓ is the potential at a distance x from the surface, Ψₒ is the surface potential and κ⁻¹ is the Debye length. An increase of the electrolyte concentration (NaCl) decreases the Debye length. Zeta potentials are defined as the electrostatic potentials at the position of the slipping plane which is assumed to occur just outside the fixed aqueous layer of a biocompatible nano-polymer particle. From equation (2) results equation (3)

lnΨₓ=lnΨ₀−κ·x (3)

If zeta potentials (Ψₓ) are measured in different concentrations of NaCl (0, 0.1, 0.2, 0.5, 1, 2 and 5 mM) and plotted against κ equal to 3.33·c^{1/2}, where c is the molarity of electrolytes, the increase in concentration compensates for the thickness of adsorbed polymer layers.

FIG. 3 shows the thickness of adsorbed PVA layers on biocompatible nano-polymer particles for newly prepared (white squares) as well as nebulized particles (black squares). Depicted in FIG. 3A are these values in dependence of the PVA concentration used. For newly prepared as well as for nebulized particles, the layer thickness ranges from 10 to 20 nm. This result is also confirmed by transmission electron microscopic images. For this purpose, a copper grid (for example S160-3, Plano, Wetzlar, Germany) is coated with a thin layer of a diluted biocompatible nano-polymer particle solution. Biocompatible nano-polymer particles are then dried on the grid and investigated using a transmission electron microscope (TEM, for example JEM-3020 TEM, JEOL, Eching, Germany) at an acceleration voltage of 300 kV. FIG. 3D shows a representative TEM image of a biocompatible nano-polymer particle of this invention, where the PVA layer (employed concentration during synthesis according to Embodiment 1 of 10 g/l) is clearly visible. The zeta potential, i.e. the surface charge of particles is negative for all NaCl-concentrations assessed (FIG. 3B). The straight line in FIG. 3C indicates the linear fit of experimental data.

2.3 Content of Active Agent in Biocompatible Nano-Polymer Particles of this Invention

To determine the PDE-5 inhibitor content of biocompatible nano-polymer particles prepared according to Embodiment 1, for example 1 ml of biocompatible nano-polymer particle suspension is subjected to centrifugation at 16873×g for 30 min at 25° C. After careful removal of the supernatant, the amount of unencapsulated PDE-5 inhibitor is determined. The pellets resulting from the centrifugation are freeze-dried, weighed and subsequently dissolved for example in chloroform which is suitable as solvent for PLGA and sildenafil. The non-dissolved fraction (stabilizer) is removed by centrifugation. Then, an aliquot of the organic phase is removed to determine the amount of encapsulated PDE-5 inhibitor. The concentration of the PDE-5 inhibitor is determined using UV/Vis spectroscopy with a spectrophotometer (for example Ultrospec® 3000, Pharmacia Biotech, Freiburg, Germany). The absorption all aliquots is measured at a wavelength of 291 nm. The amount of PDE-5 inhibitor (PDE5H) present in biocompatible nano-polymer particles (PLGA-BNPP) is calculated with the aid of a calibration curve and defined in the following formula (4).

$$PDE 5 Hcontent ( \% ( w \text{/} w ) ) = \frac{mass of PDE 5 H in PLGA - BNPP}{mass of PLGA - BNPP} \cdot 100 \qquad ( 4 )$$

Biocompatible nano-polymer particles of this invention are prepared with a theoretical content of 5% (w/w) of the active agent sildenafil (free base) according to Embodiment 1 with 1% PVA and characterized. The actual sildenafil content of biocompatible nano-polymer particles of this invention is in the range of 4.05±0.15% (w/w) and shown in FIG. 4 as function of the theoretical drug loading in dependence of the pH value. At pH 4 (black circles), the drug content of particles is therefore with a maximum of 2% (w/w) considerably lower than at pH 8 with a maximum of 5.5% (w/w) (black squares).

The drug content in dependence of the linear PLGA copolymer or branched P(VA-VS)-g-PLGA copolymer chosen with a theoretical drug loading of 10% is shown in FIG. 5. For linear PLGA copolymers, those biocompatible nano-polymer particles of this invention have the highest content with 5 to 5.5% (w/w) sildenafil which were prepared according to Embodiment 1 with the PLGA copolymer Resomer® RG502H (A). For branched P(VS-VA)-g-PLGA copolymers, the sildenafil content ranges from 5% to 8% (w/w), depending on the viscosity of the organic polymer solution and the polymer charge (B).

In addition to the parameters particle size and PDI (see 2.1), the sildenafil content is depicted in FIG. 2 as quotient of value before and value after nebulization. The figure shows that nebulization has no significant influence on the sildenafil content.

2.4 Active Agent Release from Biocompatible Nano-Polymer Particles of this Invention

Investigations with respect to the in vitro release of the active agent PDE-5 inhibitor are carried out in phosphate-buffered saline at a pH value of for example 7.4 for 500 minutes at 37° C. Assays are performed with biocompatible nano-polymer particles which have a theoretical PDE-5 inhibitor loading of 5% (w/w). Aliquots of biocompatible nano-polymer particle suspensions are transferred into glass tubes and diluted with medium consisting of phosphate-buffered saline (PBS) pH 7.4+0.1% sodium dodecyl sulfate (SDS). The subsequent incubation is performed at 37° C. with agitation of the aliquots. In parallel to the experimental assay, PDE-5 inhibitor is incubated alone in medium under identical conditions. A fraction is removed at pre-set time points and subjected to centrifugation. The release of PDE-5 inhibitor is calculated using the Korsmeyer-Peppas equation according to formula (5)

Mₜ/M_{∞}=k·tⁿ, (5)

wherein Mₜ/M_{∞} denotes the fraction of agent released, t denotes the release time, k is a kinetic constant characteristic for the active agent-polymer system, and n is an exponent characterizing the mechanism of active agent release.

The in vitro release of the PDE-5 inhibitor sildenafil from biocompatible nano-polymer particles of this invention is performed over a time period of up to 500 minutes (FIG. 6). The release from particles with polymer RG502H occurs over a period of up to 90 minutes, the release from particles with polymer P(VS-VA)-g-PLGA 8-10 occurs over a time period of up to 500 minutes; the release time from other particles of this invention with polymers P(VS-VA)-g-PLGA 2-10, P(VS-VA)-g-PLGA 4-10 and P(VS-VA)-g-PLGA 6-10 is in a range between 90 and 500 minutes (FIG. 6). During this time period, >95% sildenafil is released from particles of this invention. A nebulization with Aeroneb® Professional has no influence on the sildenafil release rate. Likewise, a spray drying performed after the preparation process according to Embodiment 1.1 has no influence on the sildenafil release kinetics (FIG. 7; black circles (with spray drying) compared to white circles (without spray drying)).

Alternatively, investigations with respect to the release from particles of this invention which were prepared via spray drying according to Embodiment 1.2 were carried out with a sildenafil content of 6% in phosphate-buffered saline at a pH-value of for example 7.4 and addition of 0.1% sodium dodecyl sulfate (SDS) for 700 minutes at 37° C. Aliquots are removed at time points as indicated in FIG. 7 and subjected to centrifugation. The cumulative release of sildenafil is determined via UV/Vis-spectroscopy as described under item 2.3.

If biocompatible particles of this invention are prepared via spray drying according to Embodiment 1.2 (RG502H particles), the active agent sildenafil is released over a time period of up to 480 minutes (FIG. 7; black triangles), while particles prepared according to Embodiment 1.1 (white circles) with alternative subsequent spray drying (composite particles; black circles) release sildenafil over a time period of up to 90 minutes (FIG. 7).

### FIGURE LEGENDS

FIG. 1

Size distribution of biocompatible nano-polymer particles of this invention, which is determined by dynamic light scattering (DLS). The black line indicates the density distribution of particle sizes, the dashed line represents the cumulative distribution of particle sizes.

FIG. 2

Stability of biocompatible nano-polymer particles of this invention during nebulization with Aeroneb® Professional. The stability is shown as ratio of final to initial properties of particles of this invention (property_{f}/propertyᵢ) (A) (PDI=polydispersity index). Fractions of biocompatible nano-polymer particle suspensions are collected during nebulization for an analysis of the stability during the nebulization process. Values are given as the mean±standard deviation (n=4).

FIG. 3

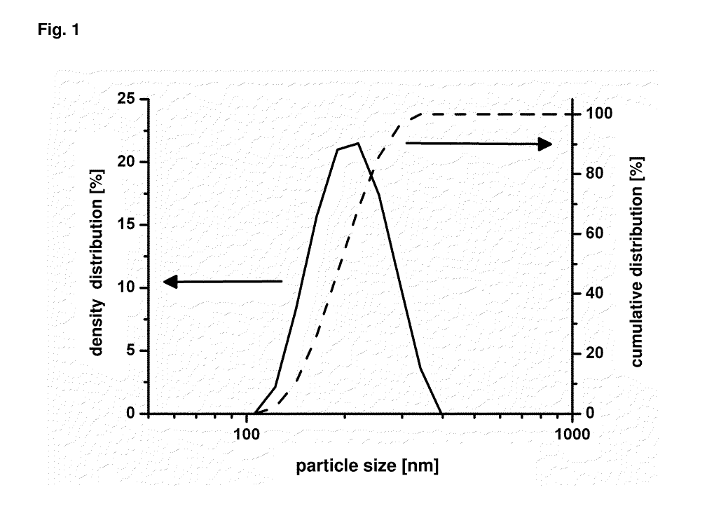

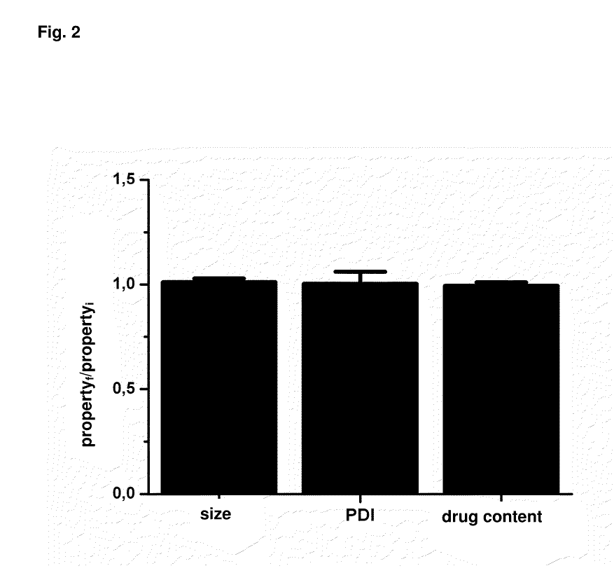

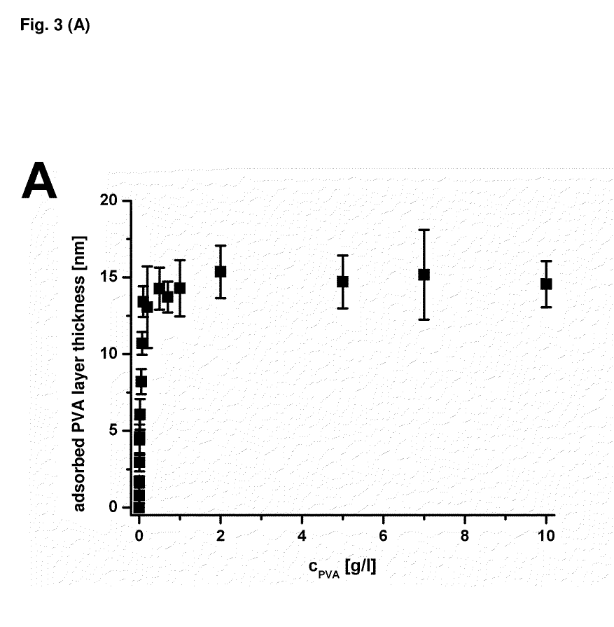

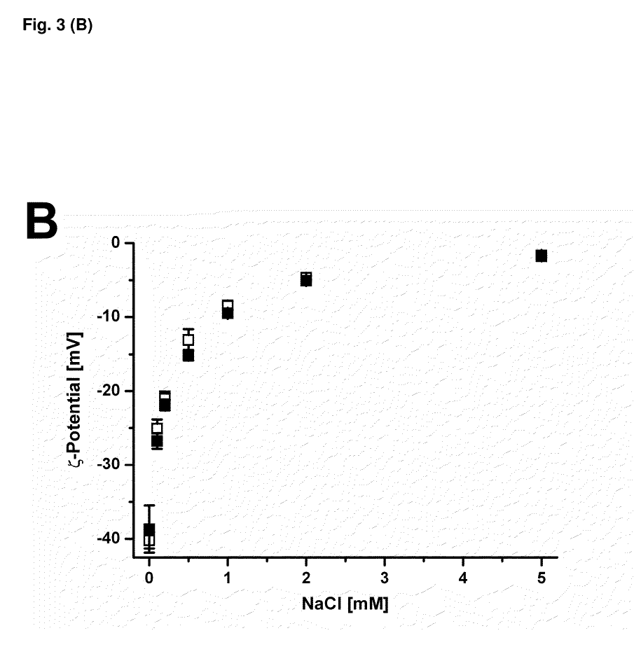

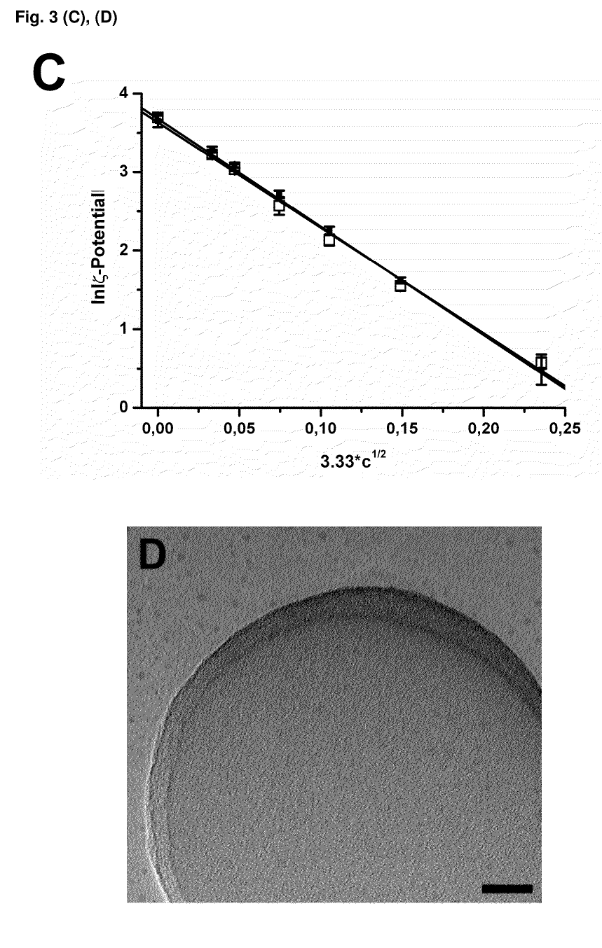

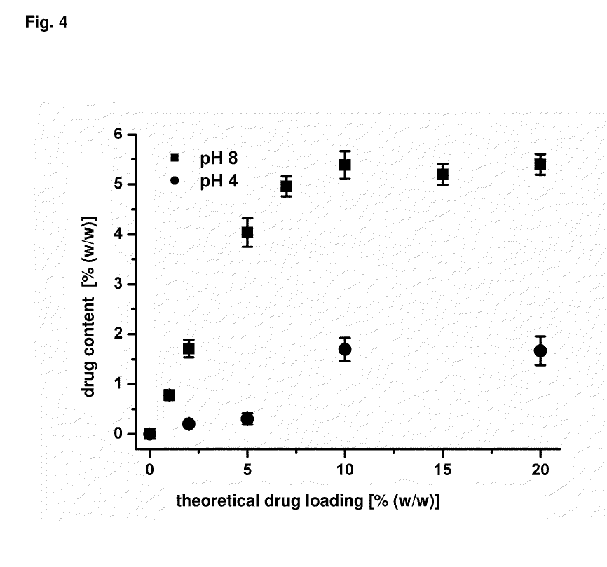

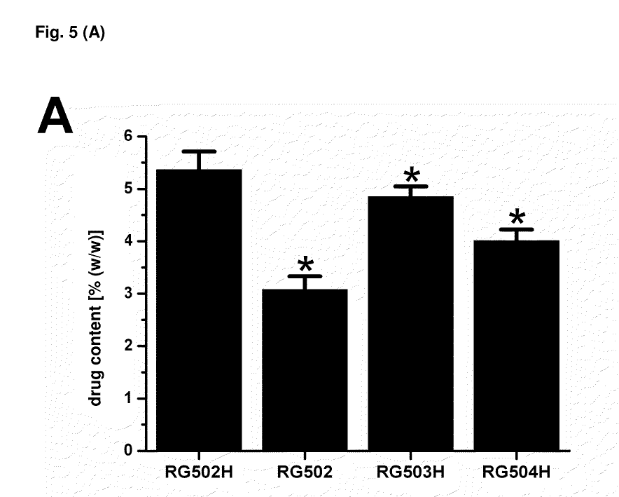

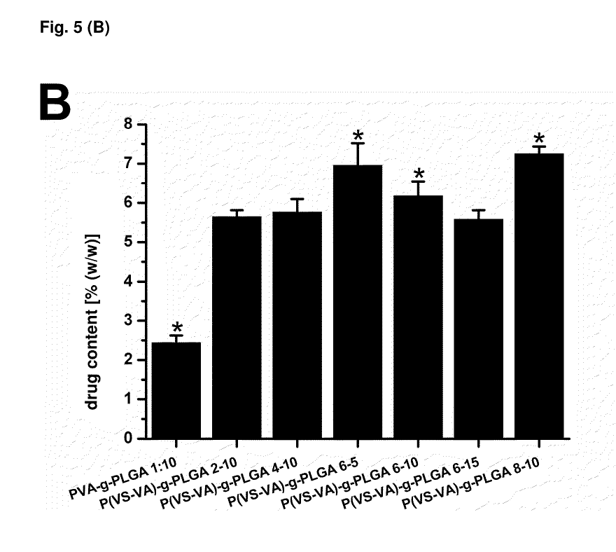

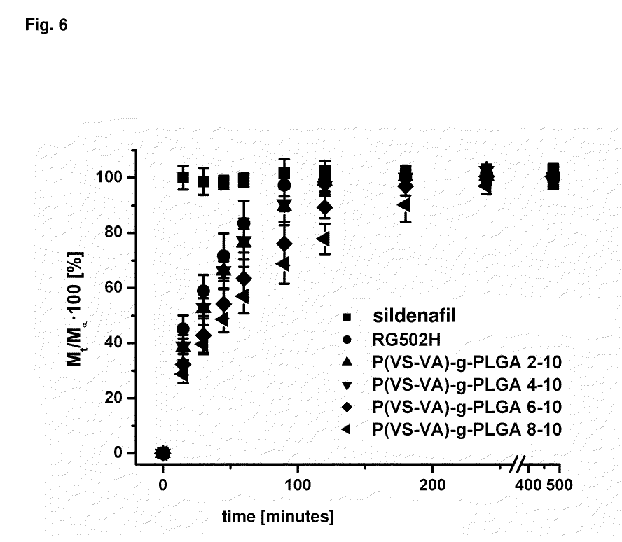

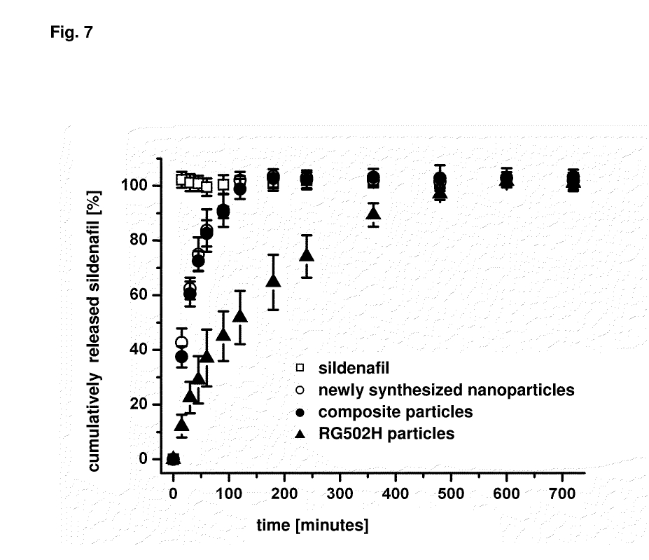

Adsorbed polyvinyl alcohol (PVA) layers thickness on biocompatible nano-polymer particles of this invention as a function of PVA concentration (c_{PVA}) (A), and zeta potential of biocompatible nano-polymer particles prepared in PVA solution as a function of electrolyte concentration (B). The slope (κ) of the In|zeta potential| versus 3.33*c^{1/2} (concentration) gives the thickness of adsorbed polymer layers (C). White and black squares in (B) and (C) represent the properties of freshly prepared (B) or nebulized (C) biocompatible nano-polymer particles, respectively. The straight line in (C) represents the linear fit of the experimental data (R²>0.99). The adsorbed PVA layer is clearly visible in the representative transmission electron microscopic image (D) (scale bar=20 nm). Values are given as the mean±standard deviation (n=4).

FIG. 4

Sildenafil content of biocompatible nano-polymer particles of this invention synthesized with 1% PVA according to Embodiment 1 at different pH values (circles=pH 4; squares=pH 8). The sildenafil content is shown in dependence on the theoretical sildenafil loading. Values are given as the mean±standard deviation (n=4).

FIG. 5

Sildenafil content of biocompatible nano-polymer particles of this invention synthesized with different linear PLGA copolymers (RG502H, RG502, RG503H or

RG504H) (A) or branched (P(VS-VA)-g-PLGA) copolymers (B) according to Embodiment 1 with a theoretical sildenafil loading of 10%. Values are given as the mean±standard deviation (n=4). Asterisks above bars indicate statistically significant differences compared with PLGA copolymer RG502H (p<0.05).

FIG. 6

In vitro sildenafil release profile of biocompatible nano-polymer particles of this invention. Fractions of biocompatible nano-polymer particle suspensions are collected during nebulization to assess the influence of nebulization on the sildenafil release profile of biocompatible nano-polymer particles. PLGA- or P(VS-VA)-g-PLGA copolymers used are each represented by different symbols as described in the figure legend. Added for comparison is the dissolution profile of free sildenafil (black squares). Values are given as the mean±standard deviation (n=4).

FIG. 7

Cumulative in vitro sildenafil release from particles prepared with spray drying (RG502H particles; black triangles) in comparison with nanoparticles freshly prepared according to Embodiment 1.1 (white circles), as well as in comparison with nanoparticles freshly prepared according to Embodiment 1.1 with subsequent spray drying of particles from the aqueous solution (composite particles; black circles), and in comparison with free sildenafil (white squares). Values are given as the mean±standard deviation (n=3).

1. Biocompatible nano-polymer particles characterized in that they are composed of a biocompatible polymer and a stabilizer and an active agent for the treatment of pulmonary hypertension or erectile dysfunction.

2. Biocompatible nano-polymer particles according to claim 1, characterized in that the biocompatible polymer is chosen from a polyester, polyanhydride, polyorthoester, polyphosphoester, polycarbonate, polyketal, polyurea, polyurethane, block copolymer (PEG-PLGA), star polymer or comb polymer.

3. Biocompatible nano-polymer particles according to claim 2, characterized in that the polyester is poly(D,L-lactide-co-glycolide) copolymer (PLGA).

4. Biocompatible nano-polymer particles according to claim 2, characterized in that the comb polymer is poly(vinyl sulfonate-co-vinyl alcohol)-graft-poly(D,L-lactide-co-glycolide) copolymer (P(VS-VA)-g-PLGA) or sulfobutyl-polyvinyl alcohol-graft-poly(lactide-co-glycolide) copolymer (SB-PVA-g-PLGA).

5. Biocompatible nano-polymer particles according to claim 1, characterized in that the stabilizer is chosen from the group consisting of non-ionic surfactants, anionic surfactants, amphoteric surfactants or polymers.

6. Biocompatible nano-polymer particles according to claim 5, characterized in that the polymer is polyvinyl alcohol (PVA).

7. Biocompatible nano-polymer particles according to claim 1, characterized in that the active agent is chosen from the group consisting of phosphodiesterase inhibitors (PDE inhibitors) or guanylate cyclase activators or guanylate cyclase stimulators or endothelin receptor antagonists or the prostanoids.

8. Biocompatible nano-polymer particles according to claim 7, characterized in that the PDE inhibitor is the PDE-5 inhibitor sildenafil.

9. Biocompatible nano-polymer particles according to claim 8, characterized in that sildenafil is present as free base.

10. Biocompatible nano-polymer particles according to claim 1, characterized in that they are nebulizable with piezoelectric, jet- or ultrasound aerosol generators, soft-mist inhalers, metered dose inhalers or dry powder inhalers.

11. Biocompatible nano-polymer particles according to claim 1, characterized in that they
have a diameter ranging from 10 nm to 10 μm and
a stabilizing layer thickness between 0 and 50 nm and
contain between 0 and 50% (w/w) of an active agent for the treatment of pulmonary hypertension or erectile dysfunction and
release the active agent over a period of up to 48 hours.

12. Biocompatible nano-polymer particles according to claim 1, characterized in that they have a diameter between 500 nm and 5 μm to achieve a longer-lasting drug release.

13. Biocompatible nano-polymer particles according to claim 1, characterized in that they particularly preferred contain between 1 and 20% (w/w) of an active agent for the treatment of pulmonary hypertension or erectile dysfunction.

14. A method for the preparation of biocompatible nano-polymer particles according to claim 1, characterized by the steps
a) dissolving of the biocompatible polymer and the active agent in a solvent under formation of an organic phase,
b) emulsifying of the organic phase in an aqueous phase which contains a stabilizer,
c) mixing of the organic and aqueous phase, and
d) removal of the solvent and obtaining the particles in suspension.

15. A method for the preparation of biocompatible nano-polymer according to claim 1, characterized by the steps
a) dissolving of the biocompatible polymer and the active agent in a solvent under formation of an organic phase, and
b) spray drying of the organic phase.

16. A method for the preparation of biocompatible nano-polymer according to one of the claims 14 to 15, characterized in that the biocompatible polymer is a polyester, polyanhydride, polyorthoester, polyphosphoester, polycarbonate, polyketal, polyurea, polyurethane, block copolymer (PEG-PLGA), star polymer or comb polymer.

17. A method for the preparation of biocompatible nano-polymer according to one of the claims 14 to 15, characterized in that the stabilizer is a non-ionic surfactant, anionic surfactant, amphoteric surfactant or polymer.

18. A method for the preparation of biocompatible nano-polymer according to one of the claims 14 to 15, characterized in that the active agent is a phosphodiesterase inhibitor (PDE inhibitor) or guanylate cyclase activator or guanylate cyclase stimulator or endothelin receptor antagonist or a prostanoid.

19. A method of treating pulmonal hypertension comprising administering an effective amount of a biocompatible nano-polymer according to claim 1 to a subject in need thereof.

20. A method of treating erectile dysfunction comprising administering an effective amount of a of biocompatible nano-polymer according to claim 1 to a subject in need thereof.

21. A method of treating pulmonal hypertension comprising administering an effective amount of a of biocompatible nano-polymer particles according to claim 19, characterized in that the pharmaceutical composition is administered via inhalation, instillation, a bronchoscope or a therapeutic respiratory device.

22. Biocompatible nano-polymer particles according to claim 1, characterized in that they have a diameter between 50 nm and 250 nm to prevent an uptake of particles by macrophages.
